(19) United States
(12) Patent Application Publication
(10) Pub. No.: US 2026/0324998 A1
(43) Pub. Date: Oct. 22, 2026

(54) OPTICAL PROBE FOR MEASURING BLOOD FLOW, AND ENDOSCOPE

(71) Applicant: TOHOKU UNIVERSITY, Sendai-shi, Miyagi (JP)

(72) Inventors: Yoichi HAGA, Sendai-shi (JP); Yoshikatsu SAIKI, Sendai-shi (JP); Noriko TSURUOKA, Sendai-shi (JP); Shintaro KATAHIRA, Sendai-shi (JP)

(21) Appl. No.: 19/108,915

(22) PCT Filed: Jul. 12, 2023
(86) PCT No.: PCT/JP2023/025718

§ 371 Date: Mar. 5, 2025

(30) Foreign Application Priority Data
Sep. 7, 2022 (JP) 2022-141902

(51) Int. Cl. A61B 5/0275; A61B 1/00; A61B 5/026; A61B 5/06

(52) U.S. Cl. CPC A61B 5/02755; A61B 1/00167; A61B 5/0261; A61B 5/062

(57) ABSTRACT

An optical probe for measuring blood flow tube is provided with a fixing member, a first optical fiber, second optical fiber, and a reflecting member. The fixing member is inserted into the tube, and fixed to an inner peripheral surface of the tube. The first optical fiber is fixed to the fixing member. The second optical fiber is fixed to the fixing member. The reflecting member reflects light output from the first optical fiber toward a direction intersecting with a longitudinal direction of the tube, and reflects light incident from the direction intersecting with the longitudinal direction of the tube toward the second optical fiber. The fixing member includes a fitting recess into which the reflecting member is fit.

### CROSS-REFERENCE TO RELATED APPLICATIONS

This application is the U.S. National Stage entry of International Application No. PCT/JP2023/025718, filed on Jul. 12, 2023, which, in turn, claims priority to Japanese Patent Application No. 2022-141902, filed on Sep. 7, 2022, both of which are hereby incorporated herein by reference in their entireties for all purposes.

### TECHNICAL FIELD

The present invention relates to an optical probe for measuring blood flow, and an endoscope.

### BACKGROUND ART

NPL 1 discloses an optical probe for measuring blood flow (hereinafter, probe) that is provided with a tube, a fixing member that is fixed to the tube, a first optical fiber and a second optical fiber fixed to the fixing member, and a reflecting member. The reflecting member reflects light output from the first optical fiber toward a side of the probe, and a tissue (target tissue) serving as a target of blood flow measurement is irradiated with the reflected light. Moreover, the reflecting member reflects the light returned from the target tissue due to scattering toward the second optical fiber. Analyzing the light output from the first optical fiber and the light scattered in the target tissue and returned to the second optical fiber using a laser blood flow meter makes it possible to measure the amount and the like of the blood flow flowing inside the target tissue.

### Citation List

### Non Patent Literature

NPL 1: Shintaro Katahira, Shunsuke Kawamoto, Naoki Masaki, Yukihiro Hayatsu, Tadao Matsunaga, Yoichi Haga, Yoshikatsu Saiki, Oesophageal mucosal blood flow changes after thoracic endovascular stent graft implantation using a novel sensor probe, Interactive CardioVascular and Thoracic Surgery, Volume 26, Issue 3, March 2018, Pages 487-493, https://doi.org/10.1093/icvts/ivx351

### SUMMARY OF INVENTION

### Technical Problem

Meanwhile, for example, in the probe described in NPL 1, positioning of the reflecting member with respect to the first optical fiber and the second optical fiber was performed by affixing the reflecting member (flat plate-like mirror) to an oblique plane included in the fixing member. Here, the accuracy of the positioning depends on the accuracy of the manufacture of the oblique plane to which the reflecting member is affixed. Accordingly, in order to perform positioning with high accuracy, the oblique plane needs to be formed on the fixing member accurately. This has increased the efforts to be put in the manufacture of the probe.

The present invention is made in view of such circumstances, and aims to provide an optical probe for measuring blood flow and an endoscope capable of easily performing positioning of a reflecting member with respect to an optical fiber.

### Solution to Problem

In order to solve the abovementioned problems, an optical probe for measuring blood flow tube according to aspect 1 of the present invention is provided with: a tube; a fixing member that is inserted into the tube, and fixed to an inner peripheral surface of the tube; a first optical fiber that is fixed to the fixing member; a second optical fiber that is fixed to the fixing member; and a reflecting member that reflects light output from the first optical fiber toward a direction intersecting with a longitudinal direction of the tube, and light incident from the direction intersecting with the longitudinal direction of the tube toward the second optical fiber, in which the fixing member includes a fitting recess into which the reflecting member is fit.

With aspect 1 of the present invention, the reflecting member is fit into the fitting recess to allow positioning of the reflecting member with respect to the first optical fiber and the second optical fiber to be performed. Accordingly, positioning of the reflecting member with respect to the optical fibers can be easily performed.

Moreover, aspect 2 of the present invention is that, in the optical probe for measuring blood flow of aspect 1, the fixing member includes: a first housing groove that communicates with the fitting recess, the first housing groove in which the first optical fiber is housed and to which the first optical fiber is fixed; and a second housing groove that communicates with the fitting recess, the second housing groove in which the second optical fiber is housed and to which the second optical fiber is fixed.

Moreover, aspect 3 of the present invention is that, in the optical probe for measuring blood flow of aspect 1 or 2, the reflecting member is a right-angle prism.

Moreover, aspect 4 of the present invention is that, in the optical probe for measuring blood flow of any one of aspects 1 to 3, the first optical fiber and the second optical fiber are spaced in the direction intersecting with the longitudinal direction with respect to the inner peripheral surface of the tube.

Moreover, aspect 5 of the present invention is that, in the optical probe for measuring blood flow of any one of aspects 1 to 4, the optical probe for measuring blood flow is further provided with a torque coil that is inserted into the tube, is fixed to the fixing member, and extends in the longitudinal direction.

Moreover, aspect 6 of the present invention is that, in the optical probe for measuring blood flow of any one of aspects 1 to 5, the optical probe for measuring blood flow is further provided with a marker member having X-ray impermeability higher than X-ray impermeability of the fixing member.

Moreover, an endoscope according to aspect 7 of the present invention is provided with: the optical probe for measuring blood flow of any one of aspects 1 to 6; and a main body portion including a forceps port into which the optical probe for measuring blood flow is inserted.

### Advantageous Effects of Invention

With the abovementioned aspects of the present invention, it is possible to provide the optical probe for measuring blood flow and the endoscope capable of easily performing positioning of the reflecting member with respect to the optical fiber.

### BRIEF DESCRIPTION OF DRAWINGS

FIG. 1 is a plan view illustrating a probe according to a first embodiment of the present invention.

FIG. 2 is a cross sectional view along II-II line in FIG. 1.

FIG. 3 is a perspective view illustrating a fixing member according to the first embodiment of the invention.

FIG. 4 is a view illustrating one example of an endoscope in which a probe according to a second embodiment of the invention is used.

FIG. 5 is a plan view illustrating the probe according to the second embodiment of the invention.

FIG. 6 is a cross sectional view along V-V line in FIG. 5.

FIG. 7 is a perspective view illustrating a fixing member according to the second embodiment of the invention.

FIG. 8 is a view illustrating another example of an endoscope in which the probe according to the second embodiment of the invention is used.

### DESCRIPTION OF EMBODIMENTS

### First Embodiment

Hereinafter, an optical probe for measuring blood flow according to a first embodiment will be described based on the drawings.

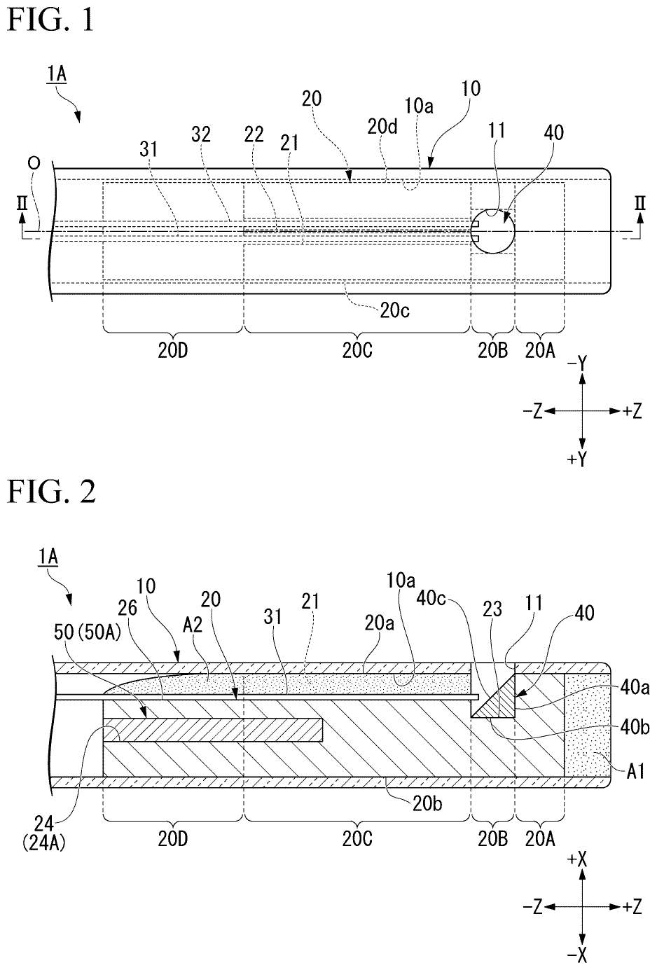

As illustrated in FIGS. 1 and 2, an optical probe for measuring blood flow 1A is provided with a tube 10, a fixing member 20, a first optical fiber 31, a second optical fiber 32, a reflecting member 40, and a marker member 50. As illustrated in FIG. 1, the tube 10 includes a central axis line O. The first optical fiber 31 and the second optical fiber 32 extend in a direction parallel to the central axis line O of the tube 10. As illustrated in FIG. 2, the first optical fiber 31 and the reflecting member 40 are opposed to each other in a direction along which the first optical fiber 31 extends (direction parallel to the central axis line O of the tube 10). Hereinafter, the optical probe for measuring blood flow 1A is simply referred to as “probe 1A” in some cases.

### Direction Definition

Here, in the present embodiment, an orientation from the first optical fiber 31 toward the reflecting member 40 in parallel to the central axis line O of the tube 10 is referred to as a front side or a distal end side, and is represented as an orientation of +Z in the drawings. An orientation opposite to the front side is referred to as a rear side or a proximal end side, and is represented as an orientation of −Z in the drawings. The front side (orientation of +Z) and the rear side (orientation of −Z), when not specially distinguished, are referred to as a longitudinal direction Z or an axial direction Z. A cross section perpendicular to the longitudinal direction Z is referred to as a transverse cross section. A direction orthogonal to the central axis line of the tube 10 is referred to as a radial direction, when viewed from the longitudinal direction Z. An orientation approaching the central axis line O along the radial direction is referred to as a radially inner side, and an orientation apart from the central axis line O is referred to as a radially outer side. A direction circling around the central axis line O is referred to as a circumferential direction, when viewed from the longitudinal direction Z. Moreover, one orientation orthogonal to the longitudinal direction Z is referred to as a left side, and is represented as an orientation of +Y in the drawings. An orientation opposite to the left side is referred to as a right side, and is represented as an orientation of −Y in the drawings. The left side (orientation of +Y) and the right side (orientation of −Y), when not specially distinguished, are referred to as a first direction Y. A cross section perpendicular to the first direction Y is referred to as a vertical cross section. One orientation orthogonal to both of the longitudinal direction Z and the first direction Y is referred to as an upper side, and is represented as an orientation of +X in the drawings. An orientation opposite to the upper side is referred to as a lower side, and is represented as an orientation of −X in the drawings. The upper side (orientation of +X) and lower side (orientation of −X), when not specially distinguished, are referred to as a second direction X.

The tube 10 is a tubular elastic member that extends in the longitudinal direction Z. The material of the tube 10 is preferably a material having high biocompatibility. Specifically, the tube 10 according to the present embodiment is a so-called nasogastric tube. As illustrated in FIGS. 1 and 2, a distal end (front end) of the tube 10 is subjected to chamfering. This prevents the tube 10 from damaging a tissue in a living body. An outer diameter of the tube 10 is, for example, approximately 14 to 18 Fr (4.7 to 6.0 mm). An inner diameter of the tube 10 is, for example, approximately 3.1 to 3.9 mm.

As illustrated in FIGS. 1 and 2, a light transmitting portion 11 is provided to a site that is opposed to the reflecting member 40 in the radial direction (the second direction X in the present embodiment), in the tube 10. The light transmitting portion 11 is configured to allow both of light to be output from the first optical fiber 31 and light to be incident on the second optical fiber 32 to pass therethrough. In the present embodiment, the light transmitting portion 11 is a through hole that opens in an outer peripheral surface of the tube 10 and extends to an inner peripheral surface 10a of the tube 10. Further, the light transmitting portion 11 does not need to be a through hole. The light transmitting portion 11 may be a site made of a transparent raw material (resin or the like), for example.

As illustrated in FIG. 2, the fixing member 20 is inserted into the tube 10, and fixed to the inner peripheral surface 10a of the tube 10. Specifically, the fixing member 20 is fixed to the inner peripheral surface 10a of the fixing member 20 by an elastic restoring force of the tube 10 toward the radially inner side and an adhesive force (details are described later) of an adhesive A1. The fixing member 20 has functions of holding the optical fibers 31 and 32 and the reflecting member 40, and performing positioning of the reflecting member 40 with respect to the optical fibers 31 and 32.

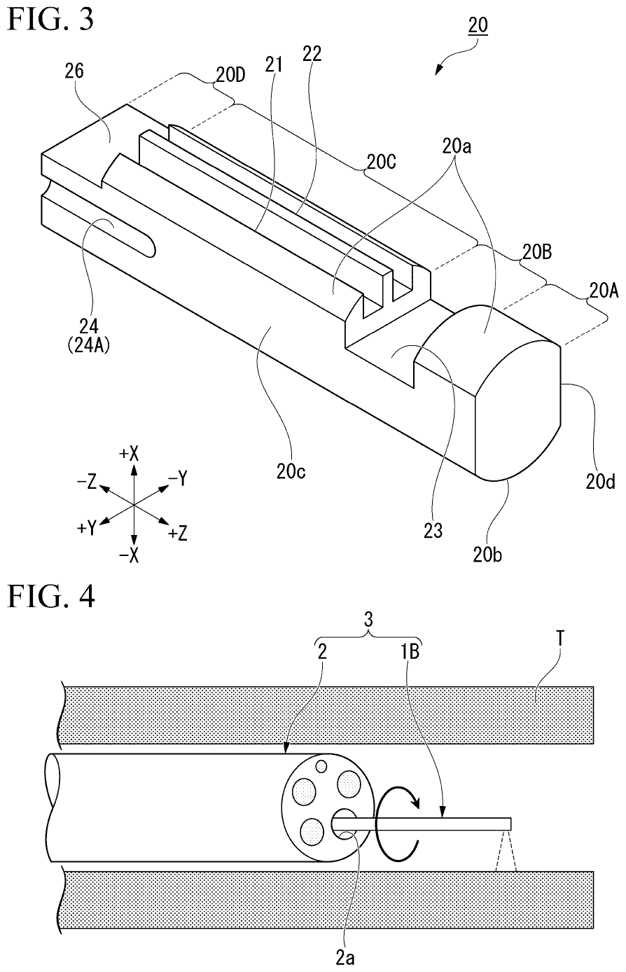

As illustrated in FIG. 3, an outer peripheral surface (surface facing the radially outer side) of the fixing member 20 according to the present embodiment includes a first curved surface 20a, a second curved surface 20b, a first flat surface 20c, and a second flat surface 20d (also see FIGS. 1 and 2). As illustrated in FIG. 3, the first curved surface 20a, the first flat surface 20c, the second curved surface 20b, and the second flat surface 20d are arranged in this order in the circumferential direction. The first curved surface 20a and the second curved surface 20b have circular arc shapes in a transverse cross sectional view. The first curved surface 20a faces the upper side. The second curved surface 20b faces the lower side. The first flat surface 20c and the second flat surface 20d have linear shapes that extend in the second direction X in a transverse cross sectional view. The first flat surface 20c faces the left side. The second flat surface 20d faces the right side.

As illustrated in FIGS. 2 and 3, the fixing member 20 according to the present embodiment includes a distal end portion 20A, a fitting portion 20B, a housing portion 20C, and a rear end portion 20D. The distal end portion 20A, the fitting portion 20B, the housing portion 20C, and the rear end portion 20D are arranged in this order in the orientation (orientation of −Z) from the front side toward the rear side, and are adjacent to each other. Note that, in the present embodiment, the distal end portion 20A, the fitting portion 20B, the housing portion 20C, and the rear end portion 20D are integrally formed of the same raw material. The material of the fixing member 20 (the distal end portion 20A, the fitting portion 20B, the housing portion 20C, and the rear end portion 20D) is preferably a material having high biocompatibility. As the material of the fixing member 20, for example, a medical polyether ether ketone (PEEK) resin or the like can be used.

As illustrated in FIGS. 2 and 3, the fitting portion 20B is a site where a fitting recess 23 is formed, in the fixing member 20. As illustrated in FIG. 2, the fitting recess 23 according to the present embodiment has a rectangular shape in a vertical cross sectional view. The reflecting member 40 is fit into the fitting recess 23. As illustrated in FIG. 3, the fitting recess 23 according to the present embodiment is open in the first curved surface 20a, the first flat surface 20c, and the second flat surface 20d.

As illustrated in FIGS. 2 and 3, the housing portion 20C is a site where a first housing groove 21 and a second housing groove 22 are formed, in the fixing member 20. As illustrated in FIGS. 1 and 2, the first optical fiber 31 is housed in the first housing groove 21. Similarly, the second optical fiber 32 is housed in the second housing groove 22. As illustrated in FIGS. 1 and 3, each of the first housing groove 21 and the second housing groove 22 is a groove that opens in the first curved surface 20a and extends in the longitudinal direction Z. The first housing groove 21 and the second housing groove 22 are spaced in the first direction Y. The first housing groove 21 is positioned on the left side (+Y side) of the second housing groove 22. Moreover, a front end of the first housing groove 21 and a front end of the second housing groove 22 communicate with the fitting recess 23.

As illustrated in FIGS. 2 and 3, the rear end portion 20D is a site that is positioned on the rear side of the housing portion 20C (the first housing groove 21 and the second housing groove 22), in the fixing member 20. In the rear end portion 20D according to the present embodiment, a cut-out recess 26 that opens in the first curved surface 20a, the first flat surface 20c, the second flat surface 20d, and a rear surface of the fixing member 20, is formed. The cut-out recess 26 communicates with a rear end of the first housing groove 21 and a rear end of the second housing groove 22.

As illustrated in FIGS. 2 and 3, the distal end portion 20A is a site that is positioned on the front side of the fitting portion 20B (the fitting recess 23), in the fixing member 20. In the distal end portion 20A according to the present embodiment, neither of the above-mentioned recess nor groove is formed. A front surface of the fixing member 20 (the distal end portion 20A) is positioned on the rear side of a distal end of the tube 10. The adhesive Al is filled in a space that is surrounded by the inner peripheral surface 10a of the tube 10 and the front surface of the fixing member 20 (the distal end portion 20A). The adhesive A1 bonds the fixing member 20 (the distal end portion 20A) to the inner peripheral surface 10a of the tube 10. As the adhesive A1, an adhesive having high biocompatibility is preferably used.

As illustrated in FIGS. 2 and 3, in the fixing member 20 according to the present embodiment, a pair of marker recesses 24 including a first marker recess 24A and a second marker recess 24B are formed (only the first marker recess 24A is illustrated). As illustrated in FIG. 3, the first marker recess 24A according to the present embodiment opens in a rear surface of the fixing member 20 and the first flat surface 20c. Although the detailed illustration is omitted, the second marker recess 24B according to the present embodiment opens in the rear surface of the fixing member 20 and the second flat surface 20d. Each of the marker recesses 24A and 24B houses the marker member 50 (see FIG. 2). Moreover, in the present embodiment, each of the marker recesses 24A and 24B extends toward the front side from the rear end of the fixing member 20 (the rear end portion 20D), and a distal end of each of the marker recesses 24A and 24B is positioned in the housing portion 20C (see FIGS. 2 and 3).

The first optical fiber 31 includes a core (not illustrated), and a clad (not illustrated) that covers the core. Similarly, the second optical fiber 32 also includes a core and a clad. Although the illustration is omitted, a laser blood flow meter is connected to proximal ends of the respective optical fibers 31 and 32 according to the present embodiment. The first optical fiber 31 propagates light input from the laser blood flow meter to a distal end of the first optical fiber 31, and outputs the light toward the front side from the distal end of the first optical fiber 31. Moreover, the second optical fiber 32 propagates light input from a distal end of the second optical fiber 32 toward a proximal end of the second optical fiber 32, and inputs the light into the laser blood flow meter.

As mentioned in the foregoing, the first optical fiber 31 is housed in the first housing groove 21 of the fixing member 20 (see FIG. 1). The second optical fiber 32 is housed in the second housing groove 22 of the fixing member 20 (see FIG. 1). As illustrated in FIG. 2, the first optical fiber 31 is fixed to the first housing groove 21 with an adhesive A2 filled in the first housing groove 21. Similarly, the second optical fiber 32 is fixed to the second housing groove 22 with the adhesive A2 filled in the second housing groove 22. As the adhesive A2, an adhesive having high biocompatibility is preferably used.

Moreover, as illustrated in FIG. 2, the first optical fiber 31 is spaced in the direction (radial direction) intersecting with the longitudinal direction Z with respect to the inner peripheral surface 10a of the tube 10. In other words, in the direction (radial direction) intersecting with the longitudinal direction Z, the space is present between the first optical fiber 31 and the inner peripheral surface 10a of the tube 10. Furthermore, the first optical fiber 31 does not come into contact with the inner peripheral surface 10a of the tube 10. Although the detailed illustration is omitted, the second optical fiber 32 is also spaced in the direction (radial direction) intersecting with the longitudinal direction Z with respect to the inner peripheral surface 10a of the tube 10. In other words, in the direction (radial direction) intersecting with the longitudinal direction Z, the space is present between the second optical fiber 32 and the inner peripheral surface 10a of the tube 10. Furthermore, the second optical fiber 32 does not come into contact with the inner peripheral surface 10a of the tube 10. Moreover, as illustrated in FIGS. 1 and 2, the first optical fiber 31 according to the present embodiment protrudes toward the front side from the first housing groove 21, and the distal end of the first optical fiber 31 is positioned in the fitting recess 23. Similarly, the second optical fiber 32 according to the present embodiment protrudes toward the front side from the second housing groove 22, and the distal end of the second optical fiber 32 is positioned in the fitting recess 23.

The reflecting member 40 according to the present embodiment is a right-angle prism made of quartz glass. In other words, as illustrated in FIG. 2, the reflecting member 40 has a triangular prismatic shape that is a right-angled isosceles triangle in a vertical cross sectional view. Specifically, the reflecting member 40 according to the present embodiment includes a first plane 40a, a second plane 40b, and a third plane (oblique plane) 40c. The first plane 40a is a plane that extends in the second direction X in a vertical cross sectional view. The second plane 40b is a plane that extends toward the rear side from a lower end of the first plane 40a in a vertical cross sectional view. Moreover, the size of the first plane 40a in the second direction X and the size of the second plane 40b in the longitudinal direction Z are approximately equivalent to each other. Note that, the wording “approximately equivalent” includes a case where the equivalence can be regarded when a manufacturing error is removed in the present description. The third plane 40c is a plane that connects an upper end of the first plane 40a to a rear end of the second plane 40b. In a vertical cross sectional view, an angle formed by the third plane 40c and the first plane 40a and an angle formed by the third plane 40c and the second plane 40 b are both approximately 45°. Note that, in the present description, the wording “approximately 45°” includes a case where the equivalence can be regarded when a manufacturing error is removed. The third plane 40c according to the present embodiment is coated with metal. The third plane 40c functions as a plane that reflects light.

As illustrated in FIG. 2, the reflecting member 40 according to the present embodiment is fit into the fitting recess 23 such that the first plane 40a comes into contact with a front surface of the fitting recess 23, and the second plane 40b comes into contact with a lower surface of the fitting recess 23. The reflecting member 40 may be bonded and fixed to the fitting recess 23. Moreover, in a state where the reflecting member 40 is fit into the fitting recess 23 in this manner, the third plane 40c of the reflecting member 40 is opposed to the first optical fiber 31 and the second optical fiber 32 in the longitudinal direction Z. Accordingly, the reflecting member 40 (the third plane 40c) reflects light output from the first optical fiber 31 toward the direction (for example, radial direction) intersecting with the longitudinal direction Z. Moreover, the reflecting member 40 (the third plane 40c) reflects light incident from the direction (for example, radial direction) intersecting with the longitudinal direction Z toward the second optical fiber 32. In other words, the fitting recess 23 is fit into the reflecting member 40 to allow positioning of the reflecting member 40 with respect to the optical fibers 31 and 32 to be performed.

The marker member 50 is a member having X-ray impermeability higher than X-ray impermeability of the fixing member 20. Accordingly, when the probe 1A is inserted into a living body, X-ray fluoroscopy is conducted to allow a position and an orientation of the probe 1A in the living body to be checked. As a material of the marker member 50, for example, platinum, gold, or the like can be employed. Although the detailed illustration is omitted, the probe 1A according to the present embodiment is provided with a first marker member 50A housed in the first marker recess 24A, and a second marker member 50B housed in the second marker recess 24B. The first marker member 50A is bonded and fixed to the first marker recess 24A. The second marker member 50B is bonded and fixed to the second marker recess 24B. Note that, the probe 1A may be provided with one, or three or more marker members 50. Alternatively, the probe 1A does not need to be provided with the marker member 50.

Next, effects by the probe 1A configured as in the foregoing will be described.

In a case where the probe 1A according to the present embodiment is used to measure an amount of blood flow in a target tissue (for example, a gastrointestinal tract or the like) in the living body, the above-mentioned laser blood flow meter outputs predetermined light to the first optical fiber 31. The output light propagates in the first optical fiber 31, and outputs toward the front side from the distal end of the first optical fiber 31. Then, the output light is reflected radially outward by the third plane 40c of the reflecting member 40, passes through the light transmitting portion 11, and the target tissue outside the probe 1A is irradiated with the output light. Further, the irradiation light is scattered in the target tissue, and returned to the probe 1A. The light scattered in the target tissue passes through the light transmitting portion 11 to enter the probe 1A, is then reflected by the third plane 40c of the reflecting member 40, and is input into the second optical fiber 32. Further, the light input into the distal end of the second optical fiber 32 propagates in the second optical fiber 32, and is input into the laser blood flow meter. The laser blood flow meter analyzes the light output to the first optical fiber 31 and the light input from the second optical fiber 32 to allow the amount of blood flow flowing through the target tissue to be measured. Specifically, the laser blood flow meter may be configured so as to measure the blood flow amount in the target tissue by using a frequency difference and an intensity difference between the light output to the first optical fiber 31 and the light input into the second optical fiber 32.

Moreover, in the probe 1A according to the present embodiment, the reflecting member 40 is fit into the fitting recess 23 to allow positioning of the reflecting member 40 with respect to each of the optical fibers 31 and 32 to be performed. Accordingly, as compared with the configuration described in NPL 1, for example, in which a reflecting member is affixed to the oblique plane included in the fixing member, it is possible to easily perform positioning of the reflecting member 40 with respect to each of the optical fibers 31 and 32.

Moreover, when the probe 1A according to the present embodiment is assembled, firstly, the optical fibers 31 and 32, the reflecting member 40, and the marker member 50 are fixed to the fixing member 20, and thereafter the fixing member 20 may be inserted into the tube 10. Accordingly, as compared with the probe described in NPL 1, for example, it is possible to reduce efforts required for the assembly of the probe 1A. Note that, in order to reduce a contact area between the fixing member 20 and the tube 10 and suppress the friction resistance, the outer peripheral surface of the fixing member 20 is not a circular shape in a transverse cross sectional view and the first flat surface 20c and the second flat surface 20d are provided. Accordingly, the fixing member 20 is likely to be inserted into the tube 10.

As has been described in the foregoing, the probe 1A according to the present embodiment is provided with: the tube 10; the fixing member 20 that is inserted into the tube 10, and fixed to the inner peripheral surface 10a of the tube 10; the first optical fiber 31 that is fixed to the fixing member 20; the second optical fiber 32 that is fixed to the fixing member 20; and the reflecting member 40 that reflects light output from the first optical fiber 31 toward a direction intersecting with the longitudinal direction Z, and reflects light incident from the direction intersecting with the longitudinal direction Z toward the second optical fiber 32, in which the fixing member 20 includes the fitting recess 23 into which the reflecting member 40 is fit.

With this configuration, the reflecting member 40 is fit into the fitting recess 23 to allow positioning of the reflecting member 40 with respect to the first optical fiber 31 and the second optical fiber 32 to be performed. Accordingly, it is possible to easily perform positioning of the reflecting member 40 with respect to the optical fibers 31 and 32.

Moreover, the fixing member 20 includes: the first housing groove 21 that communicates with the fitting recess 23, the first housing groove 21 in which the first optical fiber 31 is housed and to which the first optical fiber 31 is fixed; and the second housing groove 22 that communicates with the fitting recess 23, the second housing groove 22 in which the second optical fiber 32 is housed and to which the second optical fiber 32 is fixed. With this configuration, it is possible to easily implement the configuration in which the reflecting member 40 is fit into the fitting recess 23 to position the reflecting member 40 with respect to the optical fibers 31 and 32.

Moreover, the reflecting member 40 is a right-angle prism. With this configuration, as compared with the configuration described in NPL 1, for example, in which a reflecting member is affixed to the oblique plane included in the fixing member, it is possible to more easily make a determination of an angle of the reflecting plane (the third plane 40c) that reflects light.

Moreover, the first optical fiber 31 and the second optical fiber 32 are spaced in the direction intersecting with the longitudinal direction Z with respect to the inner peripheral surface 10a of the tube 10. Generally, light output from an optical fiber radially diffuses toward the radially outer side as the light separates from the optical fiber. By separating the optical fibers 31 and 32 from the inner peripheral surface 10a of the tube 10, as compared with a case where the optical fibers 31 and 32 are in contact with the inner peripheral surface 10a of the tube 10, for example, it is possible to widen the range which the probe 1A irradiates with light, in other words, the range serving as a target of measurement of blood flow. For example, in a case where optical fibers having a numerical aperture of approximately 0.27 to 0.29 are used as the optical fibers 31 and 32, by setting a distance in the radial direction between the optical fibers 31 and 32 and the outer peripheral surface of the tube 10 to 2.5 mm or more, it is possible to attain a diameter of 1.63 mm or more of the blood flow measurement range in the target tissue that comes into contact with the outer peripheral surface of the tube 10.

Moreover, the probe 1A according to the present embodiment is further provided with the marker member 50 having X-ray impermeability higher than X-ray impermeability of the fixing member 20. With this configuration, when the probe 1A is inserted into a living body, by conducting X-ray fluoroscopy, it is possible to check a position and an orientation of the probe 1A in the living body.

### Second Embodiment

Next, a second embodiment will be described, and the basic configuration is similar to that of the first embodiment. Accordingly, the similar configurations are assigned with the same reference numerals and the descriptions thereof are omitted, and only different points will be described.

As illustrated in FIG. 4, a probe (optical probe for measuring blood flow) 1B according to the present embodiment is used as a component that constitutes an endoscope 3. The endoscope 3 is provided with a main body portion 2 including a forceps port 2a, and the probe 1B inserted into the forceps port 2a. The probe 1B protrudes from a distal end of the main body portion 2 to measure the blood flow in a target tissue T (for example, a gastrointestinal tract or the like) . Note that, the endoscope 3 may be provided with components (for example, a camera and the like) other than the main body portion 2 and the probe 1B.

In order to insert the probe 1B into the forceps port 2a of the main body portion 2, the outer diameter of the tube 10 according to the present embodiment is smaller than the outer diameter of the tube 10 according to the first embodiment. The inner diameter of the tube 10 according to the present embodiment is, for example, approximately 1.854 to 2.489 mm. The outer diameter of the tube 10 according to the present embodiment is, for example, approximately 1.930 to 2.591 mm. The inner diameter of the forceps port 2a is, for example, approximately 2.2 to 2.8 mm.

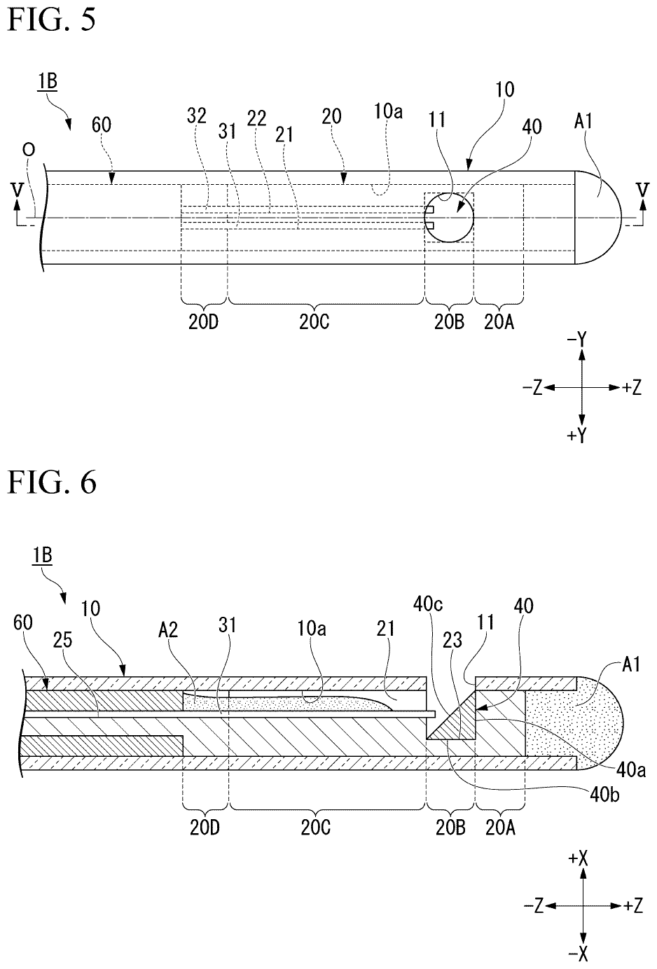

Similar to the first embodiment, the material of the tube 10 is preferably a material having high biocompatibility. As the material of the tube 10, for example, polyimide (PI), polytetrafluoroethylene (PTFE), or the like can be used. Moreover, in the present embodiment as illustrated in FIGS. 5 and 6, the adhesive A1 that bonds the fixing member 20 to the tube 10 protrudes in a hemispherical shape from the distal end of the tube 10. This prevents the distal end of the tube 10 (distal end of the probe 1B) from damaging a tissue in a living body.

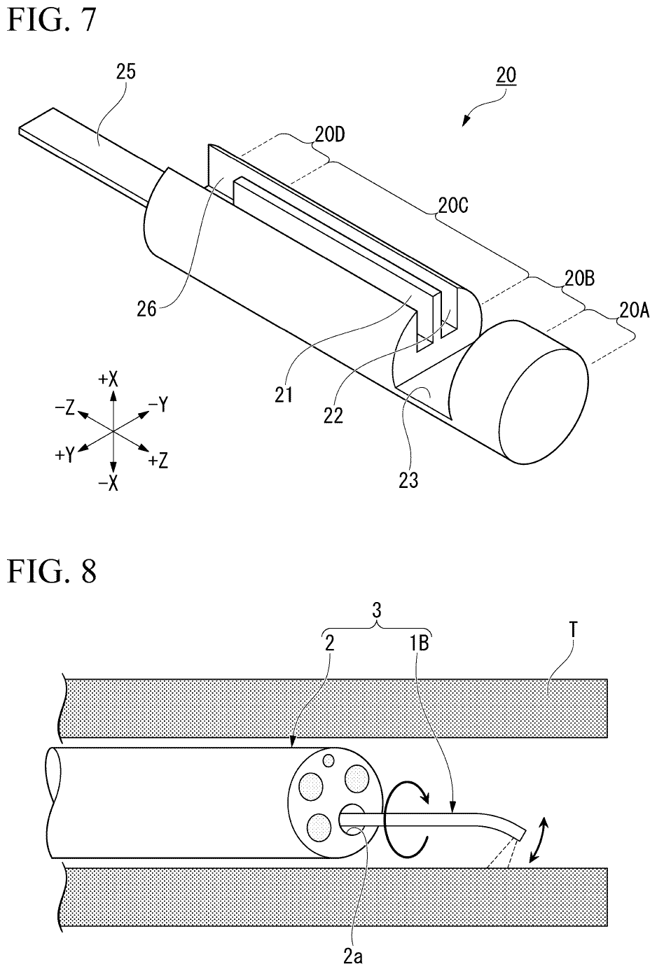

As illustrated in FIGS. 5 and 6, the probe 1B according to the present embodiment is not provided with the marker member 50, but provided with a torque coil 60 inserted into the tube 10. Moreover, the shape of the fixing member 20 is different between the probe 1B according to the present embodiment and the probe 1A according to the first embodiment. Specifically, as illustrated in FIG. 7, the outer peripheral surface of the fixing member 20 according to the present embodiment includes no flat surface, unlike the first embodiment, and is a circular shape in a transverse cross sectional view. Moreover, the fixing member 20 according to the present embodiment includes an extending portion 25 that extends toward the rear side from the rear surface of the fixing member 20. As illustrated in FIG. 6, the extending portion 25 is inserted into an inside of the torque coil 60. The torque coil 60 is fixed to the rear end portion 20D of the fixing member 20 by a holding force between the inner peripheral surface 10a of the tube 10 and the extending portion 25 and an adhesive force of the adhesive A2.

The torque coil 60 is a tubular member that extends in the longitudinal direction Z. The torque coil 60 is a member having the rigidity higher than the rigidity of the tube 10, and is more excellent in torque transmission force and rotational synchronization than the tube 10. Here, “torque transmission force” is an index, when torque around the central axis line applied to a proximal end of the probe 1B is transmitted to a distal end of the probe 1B, indicating the difficulty of attenuation of the torque. Moreover, “rotational synchronization” is an index indicating the degree of match between a rotation angle at the proximal end of the probe 1B and a rotation angle at the distal end of the probe 1B. Although the detailed illustration is omitted, the torque coil 60 according to the present embodiment has a structure in which three coils made of stainless steel is concentrically laminated in three layers. In the torque coil 60, the winding direction of the coil in the most inner layer and in the most outer layer and the winding direction of the coil in the intermediate layer are mutually reverse directions.

By connecting the torque coil 60 excellent in torque transmission force and rotational synchronization to the rear end of the fixing member 20, even in a case where the tube 10 is fine (thin) and soft, the movement of the probe 1B can be easily controlled. In other words, an operation added by a human being such as a medical doctor (or a machine such as a medical robot) to the proximal end of the probe 1B is accurately transmitted to the distal end of the probe 1B to cause the distal end of the probe 1B to be likely to perform a desired movement.

Here, also in the probe 1B according to the present embodiment, similar to the probe 1A according to the first embodiment, the reflecting member 40 is fit into the fitting recess 23 to allow positioning of the reflecting member with respect to the first optical fiber 31 and the second optical fiber 32 to be performed. Accordingly, also in the probe 1B according to the present embodiment, it is possible to easily perform positioning of the reflecting member 40 with respect to the optical fibers 31 and 32.

Moreover, as mentioned above, the probe 1B according to the present embodiment is further provided with the torque coil 60 that is inserted into the tube 10, is fixed to the fixing member 20, and extends in the longitudinal direction Z. With this configuration, even in a case where the tube 10 fine (thin) and soft, it is possible to easily control the movement of the probe 1B.

Note that, the technical range of the present invention is not limited to the embodiments, but various changes can be made within the range that does not deviate from the scope of the present invention.

For example, in the embodiments, the description has been made that the reflecting member 40 is a right-angle prism made of quartz glass, but the reflecting member 40 may be made of a transparent resin and the like. Moreover, the reflecting member 40 may be entirely formed of a metal raw material. In this case, the third plane 40c does not need to be coated with metal. Moreover, the shape of the reflecting member 40 is not limited to a triangular prism. As long as the reflecting member 40 can reflect light and the fitting recess 23 is fit into the reflecting member 40 to allow positioning of the reflecting member 40 with respect to the optical fibers 31 and 32, the material and the shape of the reflecting member 40, or the shape of the fitting recess 23 can be changed as appropriate. Similarly, the shapes of the first housing groove 21 and the second housing groove 22 can be changed as appropriate.

Moreover, the first optical fiber 31 or the second optical fiber 32 may be in contact with the inner peripheral surface 10a of the tube 10.

Moreover, as illustrated in FIG. 8, a distal end portion of the probe 1B may be configured so as to be bendable in a longitudinal direction of the endoscope 3. In this case, the probe 1B is caused to approach the target tissue T to allow the measurement of blood flow to be performed with higher accuracy.

In addition, the components in the above-mentioned embodiments can be replaced with well-known components as appropriate within the range that does not deviate from the scope of the present invention, and the above-mentioned embodiments and modifications may be combined as appropriate.

### REFERENCE SIGNS LIST

1A, 1B: probe (optical probe for measuring blood flow)
2: main body portion
2a: forceps port
3: endoscope
10: tube
10a: inner peripheral surface
20: fixing member
21: first housing groove
22: second housing groove
23: fitting recess
31: first optical fiber
32: second optical fiber
40: reflecting member
50: marker member
60: torque coil

What is claimed is:

1. An optical probe for measuring blood flow, comprising:
a tube;
a fixing member that is inserted into the tube, and fixed to an inner peripheral surface of the tube;
a first optical fiber that is fixed to the fixing member;
a second optical fiber that is fixed to the fixing member; and
a reflecting member that reflects light output from the first optical fiber toward a direction intersecting with a longitudinal direction of the tube, and light incident from the direction intersecting with the longitudinal direction of the tube toward the second optical fiber, wherein
the fixing member includes a fitting recess into which the reflecting member is fit.

2. The optical probe for measuring blood flow according to claim 1, wherein
the fixing member includes a first housing groove that communicates with the fitting recess, the first housing groove in which the first optical fiber is housed and to which the first optical fiber is fixed, and
a second housing groove that communicates with the fitting recess, the second housing groove in which the second optical fiber is housed and to which the second optical fiber is fixed.

3. The optical probe for measuring blood flow according to claim 1, wherein the reflecting member is a right-angle prism.

4. The optical probe for measuring blood flow according to claim 1, wherein, the first optical fiber and the second optical fiber are spaced in the direction intersecting with the longitudinal direction with respect to the inner peripheral surface of the tube.

5. The optical probe for measuring blood flow according to claim 1, further comprising a torque coil that is inserted into the tube, is fixed to the fixing member, and extends in the longitudinal direction.

6. The optical probe for measuring blood flow according to claim 1, further comprising a marker member having X-ray impermeability higher than X-ray impermeability of the fixing member.

7. An endoscope comprising: the optical probe for measuring blood flow according to claim 1; and a main body portion including a forceps port into which the optical probe for measuring blood flow is inserted.
